(19) United States
(12) Patent Application Publication
(10) Pub. No.: US 2019/0017028 A1
(43) Pub. Date: Jan. 17, 2019

(54) SYSTEMS AND METHODS FOR CARDIOMYOCYTE PACING

(71) Applicant: The Regents of The University of Michigan, Ann Arbor, MI (US)

(72) Inventors: Todd Herron, Ann Arbor, MI (US); Jiang Jiang, Ann Arbor, MI (US); Jose Jalife, Ann Arbor, MI (US)

(21) Appl. No.: 16/032,932

(22) Filed: Jul. 11, 2018

Related U.S. Application Data
(60) Provisional application No. 62/531,596, filed on Jul. 12, 2017.

(51) Int. Cl. C12N 5/077; C12M 1/32; C12M 1/34; C12M 1/42

(52) U.S. Cl. CPC C12N 5/0657; C12M 23/12; C12N 2506/02; C12M 35/02; C12M 41/46

(57) ABSTRACT

Provided herein are systems and methods for cardiomyocyte pacing of cultured cells, including cardiomyoctyes. In particular, provided herein are systems and methods employing electrode arrays with multiwell culture devices that provide electrical stimulation to cells cultured in individual wells of the devices.

This application claims priority to U.S. Provisional Patent Application Ser. No. 62/531,596, filed Jul. 12, 2017, which is incorporated herein by reference in its entirety.

### STATEMENT REGARDING FEDERALLY SPONSORED RESEARCH OR DEVELOPMENT

This invention was made with government support under grant HL 122352 from the National Institutes of Health. The government has certain rights in the invention.

### FIELD

Provided herein are systems and methods for cardiomyocyte pacing of cultured cells, including cardiomyoctyes. In particular, provided herein are systems and methods employing electrode arrays with multiwell culture devices that provide electrical stimulation to cells cultured in individual wells of the devices.

### BACKGROUND

Phenotype analysis of electrically excitable cells requires external electrical stimulation. For example, it has been shown that chronic stimulation of cultured cardiac myocytes prevents de-differentiation and maintains the contractile properties of the cell for longer than unstimulated cells. However, there are limited systems available for stimulating cells cultured in multi-well culture plates. For example, the eight-channel C-Pace stimulator (Ion Optix) is designed to provide stimulation to 4-well, 6-well, and 8-well dishes of particular design and configuration, but is not suitable for other configurations.

### SUMMARY

Accordingly, the technology provided herein relates to providing multiwell plate electrodes that fit to most widely used cell culture plates. In particular embodiments, the technology provides an electrode frame for 96-well plates. In particular embodiments, the technology provides an electrode frame for 24 well plates. In some embodiments, the technology finds use in point stimulation to quantify stem cell-derived cardiomyocyte action potential propagation velocity. In some embodiments, the electrode technology finds use in matching the pacing rate of stem cell-derived cardiomyocytes for drug testing, which is in contrast to current methods that are limited to only the spontaneous beating of cells is used to test drugs.

The technology provides improvements and capabilities relative to extant technologies, e.g., in some embodiments the technology provides an electrode array that is compatible with existing multiwell plates, thus abrogating the need for the manufacture of multiwell plates specifically for assay of cardiomyocytes. Embodiments of the technology find use in combination with optical mapping and can provide chronic or acute stimulation to cardiomyocytes. In some embodiments, the technology provides for the high current electrical pacing of cardiomyocytes.

Accordingly, provided herein is a technology related to an electrode array that provides electrical current or voltage to a plurality of samples in a multiwell plate, the electrode array comprising a plurality of electrodes or electrode pairs, wherein each electrode or electrode pair provides a current or a voltage to a single well of a multiwell plate. In some embodiments, the current or voltage provides an electrical stimulation to a sample, e.g., in some embodiments, each electrode or electrode pair of the electrode array provides field stimulation to a sample in a well of the multiwell plate; in some embodiments, each electrode or electrode pair of the electrode array provides point stimulation to a sample in a well of the multiwell plate. The technology is not limited in the field produced by the electrodes, e.g., in some embodiments each electrode or electrode pair of the electrode array provides approximately 5 to 10 V/cm to a sample in a well of the multiwell plate. In some embodiments, each electrode or electrode pair of the electrode array is present in a low impedance circuit (e.g., 10 ohms or less (e.g., 10, 9.5, 9, 8.5, 8, 7.5, 7, 6.5, 6, 5.5, 5, 4.5, 4, 3.5, 3, 2.5, 2, 1.5, or 1 ohms or less).

The technology is not limited in the type of sample that is stimulated with an electrical current or voltage. For example, in some embodiments the sample comprises a biological sample, e.g., a cell, tissue, organ, or organoid. In some embodiments, the sample comprises a material that responds to electrical stimulation, e.g., a biological sample that that responds to electrical stimulation, e.g., a cell, tissue, organ, or organoid that responds to electrical stimulation, e.g., in some embodiments the sample comprises a cardiomyocyte.

The technology provides an electrode array that provides electrical stimulation (e.g., a current or a voltage) to a plurality of samples, e.g., in a multiwell plate, e.g., in a multiwell plate comprising a standard configuration of well shapes and well volumes. Accordingly, in some embodiments the electrode array comprises 12 electrodes or electrode pairs. In some embodiments, the electrode array comprises 24 electrodes or electrode pairs. In some embodiments, the electrode array comprises 96 electrodes or electrode pairs.

In some embodiments, the technology provides embodiments of systems for providing electrical stimulation to a plurality of samples. For example, in some embodiments the system comprises an electrode array as described herein, e.g., an electrode array that provides electrical current or voltage to a plurality of samples in a multiwell plate, the electrode array comprising a plurality of electrodes or electrode pairs, wherein each electrode or electrode pair provides a current or a voltage to a single well of a multiwell plate; and a multiwell plate. System embodiments provide an electrode array that provides electrical stimulation (e.g., a current or a voltage) to a plurality of samples, e.g., in a multiwell plate, e.g., in a multiwell plate comprising a standard configuration of well shapes and well volumes. Accordingly, in some embodiments the electrode array is adapted to fit over the multiwell plate such that each electrode or electrode pair provides a current or a voltage to a well of the multiwell plate. In related embodiments, the system comprises a source of stimulating current or voltage, e.g., a generator, battery, or other voltage or current source. Embodiments of systems are used to test and evaluate samples that are stimulated by a current or a voltage. In some embodiments, an indicator substance (e.g., a dye; e.g., a fluorescent dye) is added to a sample (e.g., a sample is contacted with the indicator substance). Thus, in some embodiments systems comprise an indicator (e.g., a dye) for indicating changes of a sample in a well of the multiwell plate. In some embodiments, systems further comprise a source of electromagnetic radiation providing an excitation wavelength (e.g., or a range of excitation wavelengths), e.g., for exciting a fluorescent dye. In some embodiments, systems further comprise a detector of an emission wavelength of electromagnetic radiation, e.g., to detect, monitor, and/or record the intensity of emission from a sample (e.g., from an indicator dye) from a wavelength or range of wavelengths of electromagnetic radiation, e.g., to detect, monitor, and/or record an emission spectrum from a sample (e.g., from an indicator dye).

In some embodiments, systems comprise a component (e.g., hardware or software, e.g., digital or analog) for controlling the stimulating current or voltage.

In some embodiments, the systems further comprise a culture medium as described herein, e.g., a culture medium in one or more wells of a multiwell plate and, in some embodiments, comprising culture medium comprising a sample, e.g., a biological sample, in one or more wells of a multiwell plate. In some embodiments, the system comprises one or more samples, wherein each sample of the one or more samples is in a well of the multiwell plate. The systems find use in the analysis, evaluation, and/or testing of multiple samples, e.g., in a high-throughput method. Accordingly, in some embodiments the same sample is tested under different conditions and in some embodiments different samples are tested in the same conditions. For example, in some embodiments two or more samples are the same and, in some embodiments two or more samples are different. In some embodiments, the system further comprises one or more test agents, wherein each test agent of the one or more test agents is in a well of the multiwell plate. In some embodiments, the two or more test agents are the same and in some embodiments the two or more test agents are different.

Some embodiments of systems comprise cells. In some embodiments, cells are exposed to electrical stimulation using an electrode array as a component of a system as described herein. That is, in some embodiments the systems further comprise cells as described herein, e.g., cells in one or more wells of a multiwell plate and/or a culture medium comprising cells in one or more wells of a multiwell plate. In some embodiments, one or more wells of a multiwell plate comprise a biological sample that comprises cells in one or more wells of a multiwell plate. The technology is not limited in the types of cells that are a component of the system embodiments. Accordingly, in various embodiments of systems many types of cells (e.g., cultured cells, stem cells, synthetic cells) are employed. In some embodiments, systems comprise stem cells, e.g., including but not limited to embryonic stem cells and pluripotent stem cells (e.g., induced pluripotent stem cells, e.g., derived from stem cells or adult somatic cells that have undergone a dedifferentiation process), regardless of source. In some embodiments, induced pluripotent stem cells are obtained from adult human cells (e.g., fibroblasts). In some embodiments, systems comprise a pluripotent cell with potential for cardiomyocyte differentiation. In some embodiments, systems comprise cells that are cardiomyocytes, neurons, stem cell-derived cardiomyocytes (e.g., cultured and/or differentiated on a flexible (e.g., soft, pliable) surface (e.g., polydimethylsiloxane) coated with ECM proteins and/or produced using compositions and methods described in U.S. Pat. App. Pub. No. 2015/0329825, incorporated herein by reference in its entirety), stem cell-derived neurons, cells comprising ion channels, or cells comprising a proton pump. In some embodiments of systems, the cells are non-terminally differentiated cells (regardless of pluripotency) or other non-maturated cells. In some embodiments, systems comprise cells upon which a test agent acts and/or cells that are monitored for a response to a test agent. In some embodiments of systems, cells are modified to include a marker and used as diagnostic compositions to assess properties of the cells in response to changes in their environment (e.g., response to a test agent). In some embodiments, systems comprise cardiomyocytes. In some embodiments, systems comprise feeder cells.

Embodiments of the technology provide methods of providing electrical stimulation to one or more samples in parallel. For example some embodiments provide methods comprising providing one or more samples in one or more wells of a multiwell plate; contacting one or more wells of the multiwell plate with an electrode array as described herein, e.g., for providing electrical current or voltage to the one or more samples in the multiwell plate, the electrode array comprising a plurality of electrodes or electrode pairs, wherein each electrode or electrode pair provides a current or a voltage to a single well of a multiwell plate; and providing electrical stimulation to the one or more samples via the electrode array.

In related embodiments of methods, the technology provides a method of assessing the effects of electrical stimulation on one or more samples in parallel. For example, in some embodiments methods comprise providing one or more samples in one or more wells of a multiwell plate; contacting one or more wells of the multiwell plate with an electrode array as described herein, e.g., for providing electrical current or voltage to the one or more samples in the multiwell plate, the electrode array comprising a plurality of electrodes or electrode pairs, wherein each electrode or electrode pair provides a current or a voltage to a single well of a multiwell plate; providing electrical stimulation to the one or more samples via the electrode array; and observing the one or more samples stimulated by electrical stimulation. In some embodiments observing the one or more samples comprises contacting one or more samples with an indicator substance; accordingly, in some embodiments methods comprise a step of contacting one or more samples with an indicator substance, e.g., a dye, e.g., a fluorescent dye. As described herein, some embodiments comprise illuminating one or more samples with an excitation wavelength or a range of excitation wavelengths, e.g., for exciting a fluorescent dye. Some embodiments further comprise monitoring one or more samples for an emission wavelength of electromagnetic radiation (e.g., using a detector of an emission wavelength of electromagnetic radiation). Some embodiments comprise detecting, monitoring, and/or recording the intensity of emission from a sample (e.g., from an indicator dye) from a wavelength or range of wavelengths of electromagnetic radiation, e.g., detecting, monitoring, and/or recording an emission spectrum from a sample (e.g., from an indicator dye). In some embodiments, the methods comprise varying the electrical stimulation provided to the electrode array, e.g., to provide pulses of electrical stimulation, e.g., methods comprise pacing a sample. In some embodiments of methods, one or more samples comprises a cardiomyocyte and the method comprises pacing the cardiomyocyte. The technology is not limited in the frequency of pacing, e.g., in some embodiments the methods comprise varying the electrical stimulation provided to the electrode array at a frequency of 60 to 100 pulses per minute.

### BRIEF DESCRIPTION OF THE DRAWINGS

These and other features, aspects, and advantages of the present technology will become better understood with regard to the following drawings:

FIG. 1 is a drawing of an embodiment of the technology that is a 12-well chamber field pacing electrode assembly shown in both top and side views. Thin lines inside the rectangular frame indicate conductive features such as wires, electrodes, etc.

FIG. 2 is a drawing of an embodiment of the technology that is a 12-well chamber field pacing electrode assembly shown in both top and side views. Thin lines inside the rectangular frame indicate conductive features such as wires, electrodes, etc. The 12 black features in the top view and 4 black features in the side view indicate a sample (e.g., cells, monolayer of cells, 3D tissue, etc.) in the wells of the electrode assembly.

FIG. 3 is a schematic circuit drawing of an embodiment of the technology that is a 12-well chamber field pacing system. Rows A, B, and C; and columns 1, 2, 3, and 4 of the electrode array are designated. The stimulator provides a current and/or a voltage to the electrode array.

FIG. 4 is a drawing of an embodiment of the technology that is a 12-well chamber bipolar point pacing electrode assembly shown in both top and side views.

FIG. 5 is a drawing of an embodiment of the technology that is a 12-well chamber bipolar point pacing electrode assembly shown in both top and side views. The 12 black features in the top view and 4 black features in the side view indicate a sample (e.g., cells, monolayer of cells, 3D tissue, etc.) in the wells of the electrode assembly.

FIG. 6 is a schematic circuit drawing of an embodiment of the technology that is a 12-well chamber bipolar point pacing electrode assembly. Rows A, B, and C; and columns 1, 2, 3, and 4 of the electrode array are designated. The stimulator provides a current and/or a voltage to the electrode array.

FIG. 7 is a drawing of an embodiment of the technology that is a 24-well chamber field pacing electrode assembly shown in both top and side views. Thin lines inside the rectangular frame indicate conductive features such as wires, electrodes, etc.

FIG. 8 is a drawing of an embodiment of the technology that is a 24-well chamber field pacing electrode assembly shown in both top and side views. Thin lines inside the rectangular frame indicate conductive features such as wires, electrodes, etc. The 24 black features in the top view and 6 black features in the side view indicate a sample (e.g., cells, monolayer of cells, 3D tissue, etc.) in the wells of the electrode assembly.

FIG. 9 is a schematic circuit drawing of an embodiment of the technology that is a 24-well chamber field pacing system. Rows A, B, C, and D; and columns 1, 2, 3, 4, 5, and 6 of the electrode array are designated. The stimulator provides a current and/or a voltage to the electrode array.

FIG. 10 is a drawing of an embodiment of the technology that is a 24-well chamber bipolar point pacing electrode assembly shown in both top and side views.

FIG. 11 is a drawing of an embodiment of the technology that is a 24-well chamber bipolar point pacing electrode assembly shown in both top and side views. The 24 black features in the top view and 6 black features in the side view indicate a sample (e.g., cells, monolayer of cells, 3D tissue, etc.) in the wells of the electrode assembly.

FIG. 12 is a schematic circuit drawing of an embodiment of the technology that is a 24-well chamber bipolar point pacing electrode assembly. Rows A, B, C. and D; and columns 1, 2, 3, 4, 5, and 6 of the electrode array are designated. The stimulator provides a current and/or a voltage to the electrode array.

FIG. 13 is a drawing of an embodiment of the technology that is a 96-well chamber field pacing electrode assembly shown in both top and side views. Thin lines inside the rectangular frame indicate conductive features such as wires, electrodes, etc.

FIG. 14 is a drawing of an embodiment of the technology that is a 96-well chamber field pacing electrode assembly shown in both top and side views. Thin lines inside the rectangular frame indicate conductive features such as wires, electrodes, etc. The 96 black features in the top view and 12 black features in the side view indicate a sample (e.g., cells, monolayer of cells, 3D tissue, etc.) in the wells of the electrode assembly.

FIG. 15 is a schematic circuit drawing of an embodiment of the technology that is a 96-well chamber field pacing system. Rows A-H; and columns 1-12 of the electrode array are designated. The stimulator provides a current and/or a voltage to the electrode array.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein are apparatuses, devices, systems, and methods for cardiomyocyte pacing of cultured cells, including cardiomyoctyes. In particular, provided herein are systems and methods employing electrode arrays with multiwell culture devices that provide electrical stimulation to cells cultured in individual wells of the devices.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the term “functionally mature cardiomyocytes” refers to cardiomyoctes that exhibit one or more properties of primary cardiomyocytes (e.g., electrophysiological properties described herein). In some embodiments, “functionally mature cardiomyocytes” are also referred to as “electrophysiologically mature cardiomyocytes.”

“Feeder cells” or “feeders” are terms used to describe cells of one type that are co-cultured with cells of another type, to provide an environment in which the cells of the second type can grow. When a cell line spontaneously differentiates in the same culture into multiple cell types, the different cell types are not considered to act as feeder cells for each other within the meaning of this definition, even though they may interact in a supportive fashion. “Without feeder cells” refers to processes whereby cells are cultured without the use of feeder cells.

A cell is said to be “genetically altered” when a polynucleotide has been transferred into the cell by any suitable means of artificial manipulation, or where the cell is a progeny of the originally altered cell that has inherited the polynucleotide. The polynucleotide will often comprise a sequence encoding a protein of interest, which enables the cell to express the protein at an elevated level. The genetic alteration is said to be “inheritable” if progeny of the altered cell have the same alteration.

As used herein, the term “treatment” and “test treatment” are defined as the application or administration of a therapeutic agent to an isolated tissue, cell line, or cell e.g., a composition comprising one or more cardiomyocytes. In some embodiments, the treatment is applied to an isolated cardiomyocyte to evaluate the effect of the treatment on the cardiomyocyte.

As used herein, the term “test compound” refers to a chemical to be tested by one or more screening method(s). A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar, and 10 micromolar. Test compound controls can include the measurement of a signal in the absence of the test compound or comparison to a compound having a known effect.

As used herein, the term “pacing” refers to the regulation of contraction of heart muscle, cardiomyocytes, or other heart cells by the application of electrical stimulation pulses or shocks to the heart muscle, cardiomyocytes, or other heart cells.

The “electrophysiology” of a cell or tissue refers to the electrical properties of said cell or tissue. These electrical properties are measurements of voltage change or electrical current flow at a variety of scales including, but not limited to, single ion channel proteins, single cells, small populations of cells, tissues comprised of various cell populations, and whole organs (e.g., the heart). Several cell types and tissues have electrical properties including, but not limited to, muscle cells, liver cells, pancreatic cells, ocular cells, and neuronal cells. The electrical properties of a cell or tissue can be measured by the use of electrodes (examples include, but are not limited to, simple solid conductors including discs and needles, tracings on printed circuit boards, and hollow tubes, such as glass pipettes, filled an electrolyte). Intracellular recordings can be made by using techniques such as the voltage clamp, current clamp, patch-clamp, or sharp electrode. Extracellular recordings can be made by using techniques such as single unit recording, field potentials, and amperometry. A technique for high throughput analysis can also be used, such as the planar patch clamp. In another aspect, the Bioelectric Recognition Assay (BERA) can be used to measure changes in the membrane potential of cells. The above techniques are described in the following U.S. Pat. Nos. 7,270,730; 5,993,778; 6,461,860 and described in the following literature Hamill et al. (1981) Pflugers Arch. 391(2):85-100; Alvarez et al. (2002) Adv. Physiol. Educ. 26(1-4):327-341; Kornreich (2007) J. Vet. Cardiol. 9(1):25-37; Perkins (2006) J. Neurosci. Methods. 154(1-2):1-18; Gurney (2000) J. Pharmacol. Toxicol. Methods. 44(22):409-420; Baker et al. (1999) J. Neurosci. Methods 94(1):5-17: McNames and Pearson (2006) Conf. Proc. IEEE Eng. Med. Biol. Soc. 1(1): 1185-1188; Porterfield (2007) Biosens. Bioelectron. 22(7):1186-1196; Wang and Li (2003) Assay Drug Dev. Technol. 1(5):695-708; and Kintzios et al. (2001) Biosens. Bioelectron. 16(4-5):325-336.

As used herein, “stem cell” refers to a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. The term “stem cell” also includes “dedifferentiated” stem cells, an example of which is a somatic cell which is directly converted to a stem cell, e.g., the cell is “reprogrammed”. A clone is a line of cells that is genetically identical to the originating cell; in this case, a stem cell.

The term “culturing” refers to the in vitro propagation of cells or organisms on or in media of various kinds. It is understood that the descendants of a cell grown in culture may not be completely identical (e.g., morphologically, genetically, or phenotypically) to the parent cell. By “expanded” is meant any proliferation or division of cells.

The term “isolated” when used in relation to a nucleic acid (polynucleotide), peptide, polypeptide, or cell, refers to a nucleic acid (polynucleotide), peptide, polypeptide, or cell that is identified and/or separated from at least one contaminant nucleic acid, polypeptide, cell type or other biological component with which it is ordinarily associated in its natural source. Isolated polynucleotide, peptide, polypeptide, or donor cells, e.g., stem cells, are present in a form or setting that is different from that in which it is found in nature. “Purified” includes when an object species is the predominant species present (e.g., it is more abundant than any other individual species in the composition), and preferably the object species comprises at least about 50 percent of all macromolecular species present. Generally, “substantially purified” includes when an object species is more than about 80 percent of all macromolecular species present in a composition, e.g., more than about 85%, about 90%, about 95%, or about 99%.

The term “biocompatible” as used herein includes any material which upon implantation does not provoke an undesirable adverse response in a patient (e.g., an undesirable reaction other than the expected response to the trauma of implantation). When introduced into a patient, a biocompatible material is not toxic or harmful to that patient, and does not cause immunological rejection.

The term “biodegradable” as used herein refers to the ability of materials to degrade under physiological conditions to form a product that can be metabolized or excreted without damage to organs. Biodegradable materials are not necessarily hydrolytically degradable and may require enzymatic action to fully degrade. Biodegradable materials also include materials that are broken down in cells.

As used herein, “field stimulation” refers to applying electrical stimulation (e.g., a voltage and/or a current) (e.g., to cells, tissues, organs, organoids, etc.) over a general field and/or an area and a response is evoked by this electromagnetic field.

As used herein, “point stimulation” refers to stimulation that is directly applied to a specific focal point. A response is evoked by this local charge and the stimulus spreads from one excitable cell to another.

As used herein, a “bipolar electrode” (e.g., a “bipolar point pacing electrode”) is a component in which a lead (e.g., comprising wires) comprises both anode and cathode of an electrode at its tip. For example, in some embodiments, the bipolar electrode comprises a pair of coaxial electrodes, separated by an insulator. In some embodiments, the bipolar electrode comprises a pair of electrodes formed into the shape of a helix. Some embodiments comprise two parallel conductors. In a bipolar arrangement, current flows between the two electrodes at the tip of the lead.

As used herein, “low impedance” or “low resistance” refers to an impedance and/or a resistance of 10 ohms or less (e.g., 10, 9.5, 9, 8.5, 8, 7.5, 7, 6.5, 6, 5.5, 5, 4.5, 4, 3.5, 3, 2.5, 2, 1.5, or 1 ohms or less). As used herein, the terms “low impedance” and “low resistance” can refer to a circuit comprising multiple components (e.g., electrodes, wires, power supply, etc.) or a component of a circuit (e.g., an electrode). In some embodiments, impedance comprises a resistance component and a reactance component. In some embodiments, impedance and resistance are essentially, substantially, measurably, or effectively the same.

### Electrode Arrays

In some embodiments, the technology relates to an electrode array that fits over routinely used multiwell plates, e.g., to provide current and/or voltage to cardiomyocytes cultured in the wells of a multiwell plate. In some embodiments, the technology finds use in point stimulation to enable measurement of impulse propagation. In some embodiments, the technology relates to field stimulation electrodes for a multiwell plate, e.g., to match pacing frequency for testing drug effects on action potential duration.

In some embodiments, the electrodes are provided as a unipolar lead, e.g., a single conductor lead with an electrode located at the tip. In some embodiments, the electrodes are provided as a bipolar lead, e.g., comprising two separate and isolated conductors (e.g., electrodes) within a single lead. Several types of bipolar lead exist: those with two parallel conductors, the currently used coaxial leads, and the single coil type. In a bipolar arrangement, current flows between the two electrodes at the tip of the lead.

The technology provides an array of electrodes such that two electrodes are in contact with the culture medium in one or more wells of a multiwell plate, e.g., to provide a current and/or a voltage to the culture medium of the one or more wells of the multiwell plate. Providing a current and/or a voltage to the one or more wells of the multiwell plate provides a current and/or a voltage to one or more cardiomyocytes cultured in the one or more wells of the multiwell plate.

In some embodiments, each pair of two electrodes provides an electric field (E) to one or more wells of the multiwell plate that is estimated as the voltage across the electrodes (V₀) divided by the electrode gap (g), E=V₀/g. In some embodiments, the device provides an electric field of approximately 5 to 10 V/cm to the culture medium (e.g., to the one or more cultured cardiomyocytes) in the one or more wells of the multiwell plate. In some embodiments, each pair of two electrodes is present in a circuit having a low impedance (e.g., 10 ohms or less (e.g., 10, 9.5, 9, 8.5, 8, 7.5, 7, 6.5, 6, 5.5, 5, 4.5, 4, 3.5, 3, 2.5, 2, 1.5, or 1 ohms or less)). In some embodiments, the electrodes and the culture medium in contact with the electrodes are components of a circuit having a low impedance (e.g., 10 ohms or less (e.g., 10, 9.5, 9, 8.5, 8, 7.5, 7, 6.5, 6, 5.5, 5, 4.5, 4, 3.5, 3, 2.5, 2, 1.5, or 1 ohms or less)). In some embodiments, the electrode resistance is 10 ohms or less (e.g., 10, 9.5, 9, 8.5, 8, 7.5, 7, 6.5, 6, 5.5, 5, 4.5, 4, 3.5, 3, 2.5, 2, 1.5, or 1 ohms or less).

The technology is not limited in the material used for the electrodes provide that the material carries the appropriate current and/or provides the appropriate voltage to the cultured cardiomyocytes. For example, in some embodiments the electrodes are made of a noble metal (e.g., gold, platinum, palladium), a refractory metal (e.g., titanium, tungsten, molybdenum, iridium), a corrosion-resistant alloy (e.g., stainless steel), a carbon material (e.g., carbon fiber), or an organic conductor (e.g., graphite, polypyrrole). In some embodiments, the electrodes are made from silver chloride. In some embodiments, the electrode material is an electrochemical half-cell, such as a silver/silver chloride electrode. In some embodiments, a first electrode of a pair of electrodes is made of the same material as a second electrode from the pair of electrodes. In some embodiments, a first electrode of a pair of electrodes is made of a different material as a second electrode from the pair of electrodes.

In some embodiments, the device comprising the array of electrodes is reusable, e.g., for repeated use with a series of multiwell plates. In some embodiments, the device comprising the array of electrodes is sterilizable, e.g., to prevent cross-contamination from one multiwell plate assay to one or more subsequent multiwell assays.

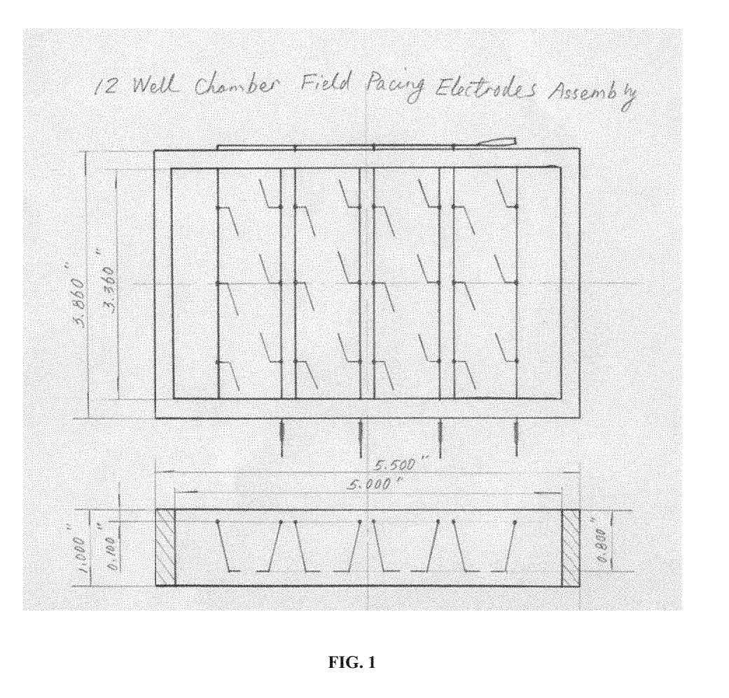

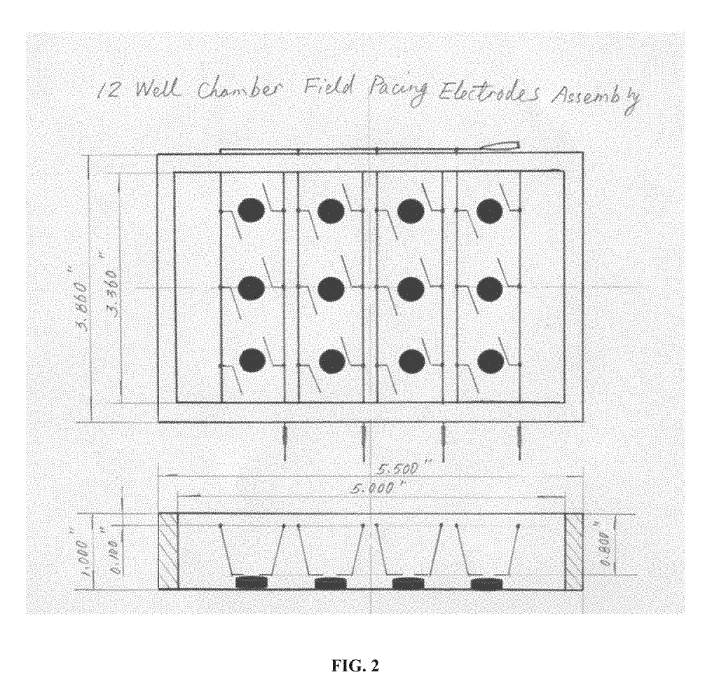

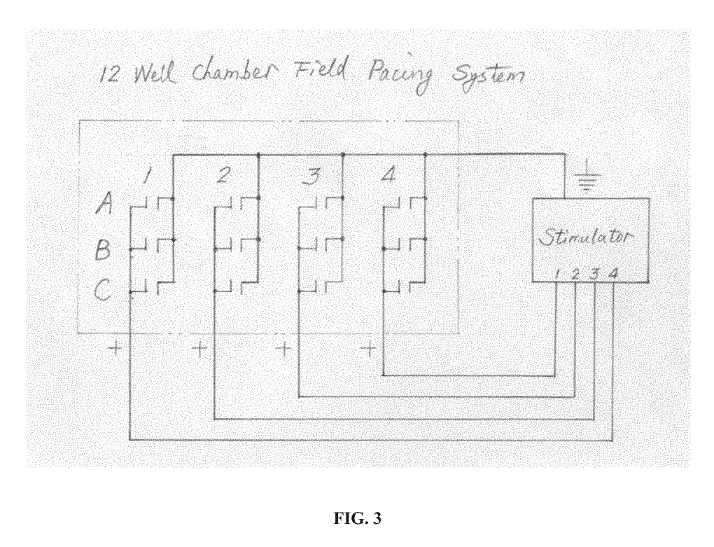

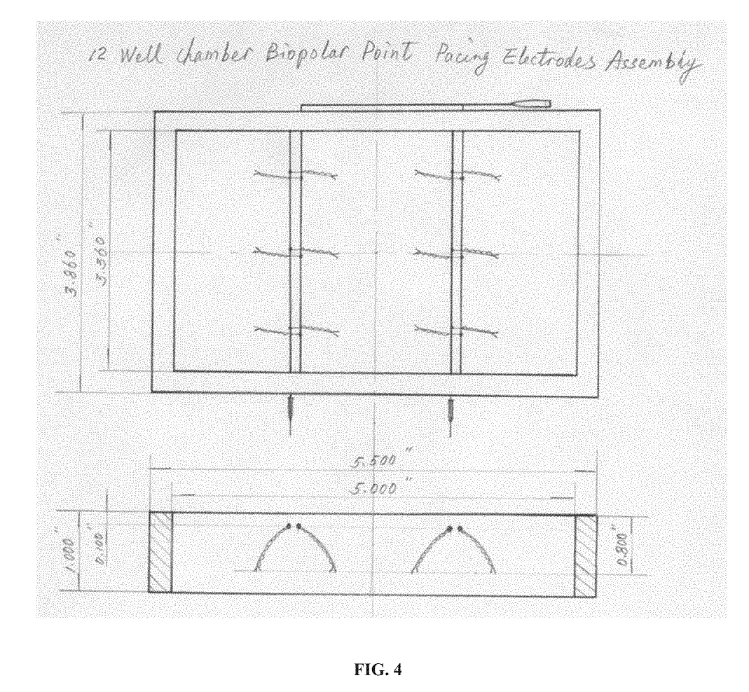

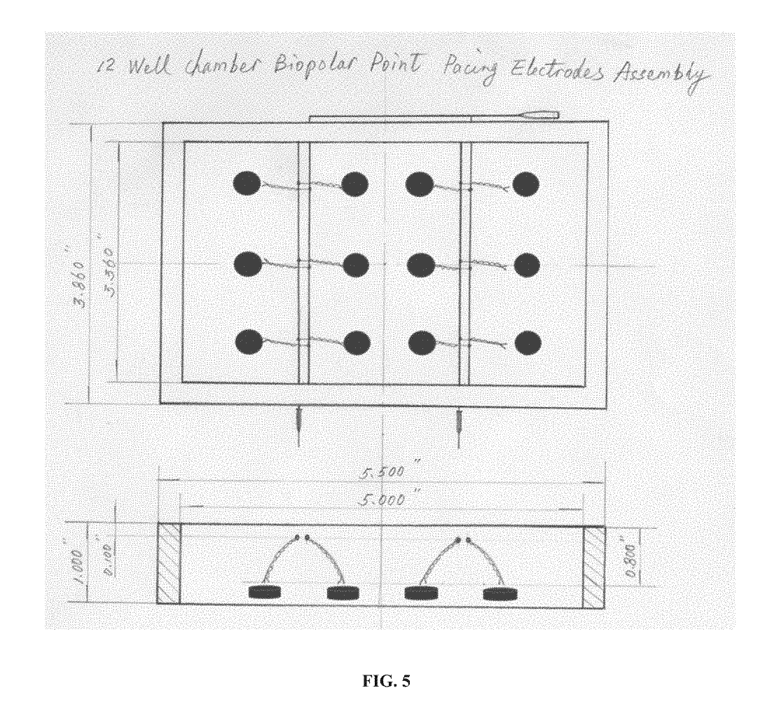

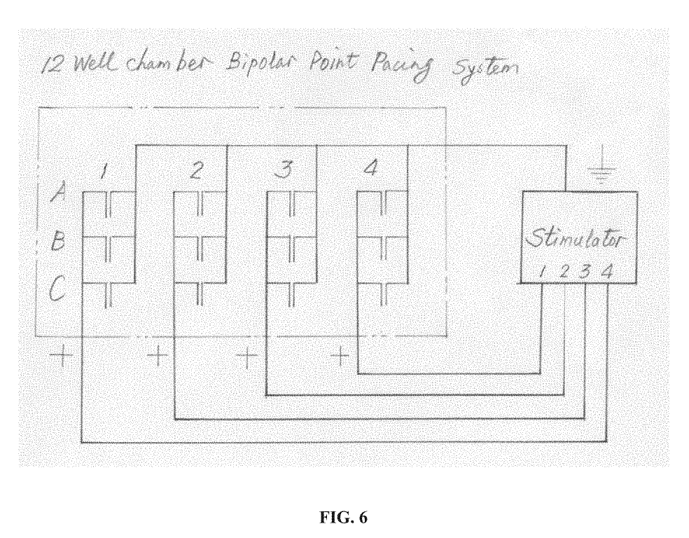

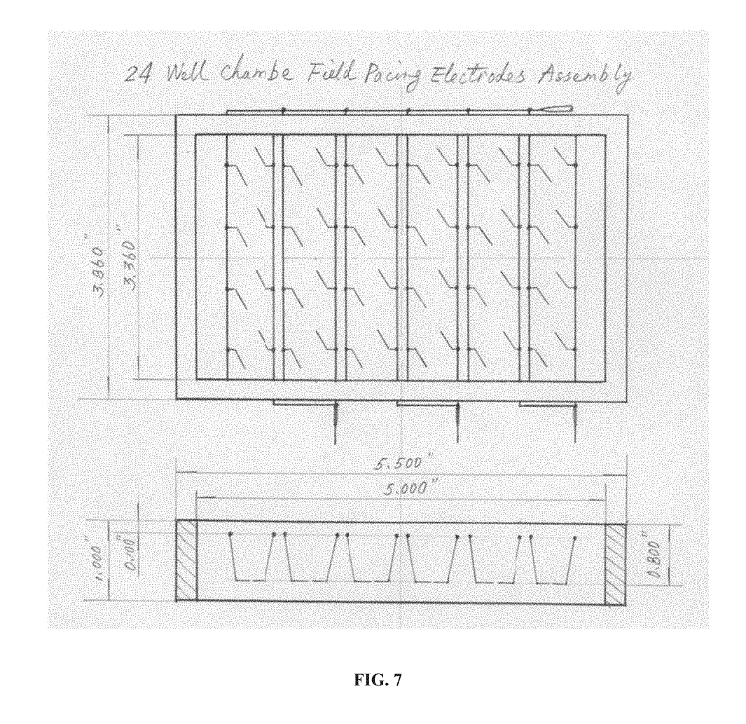

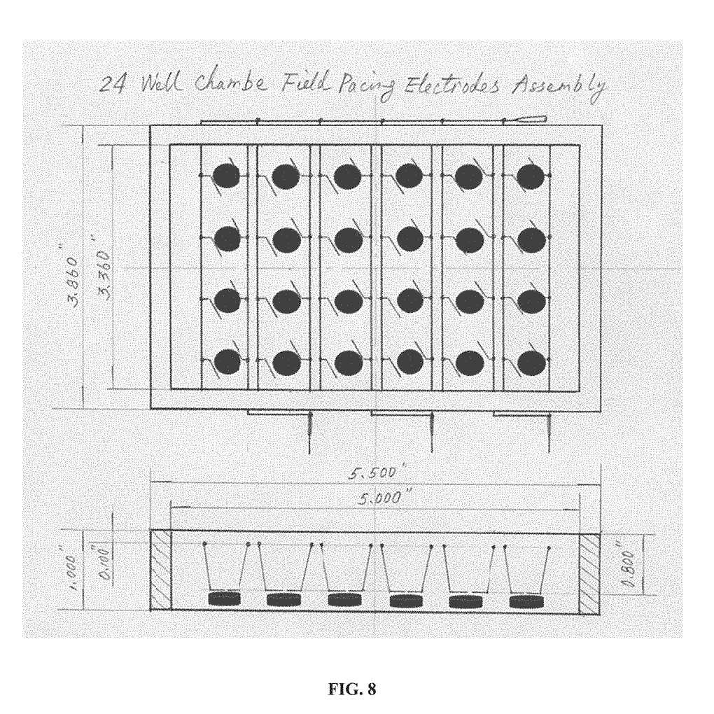

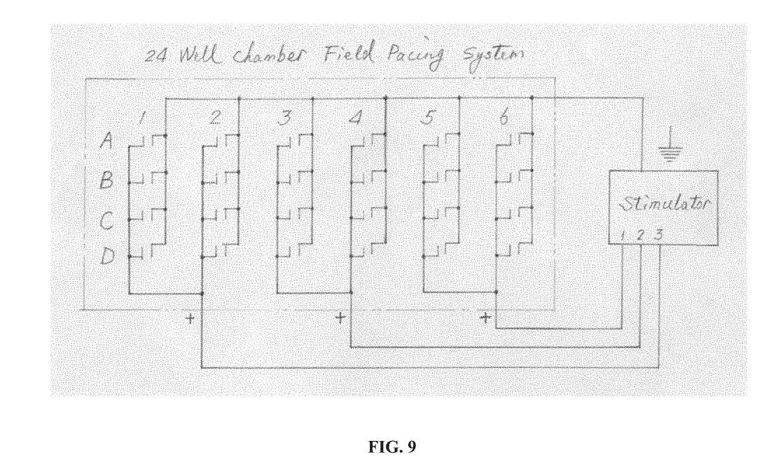

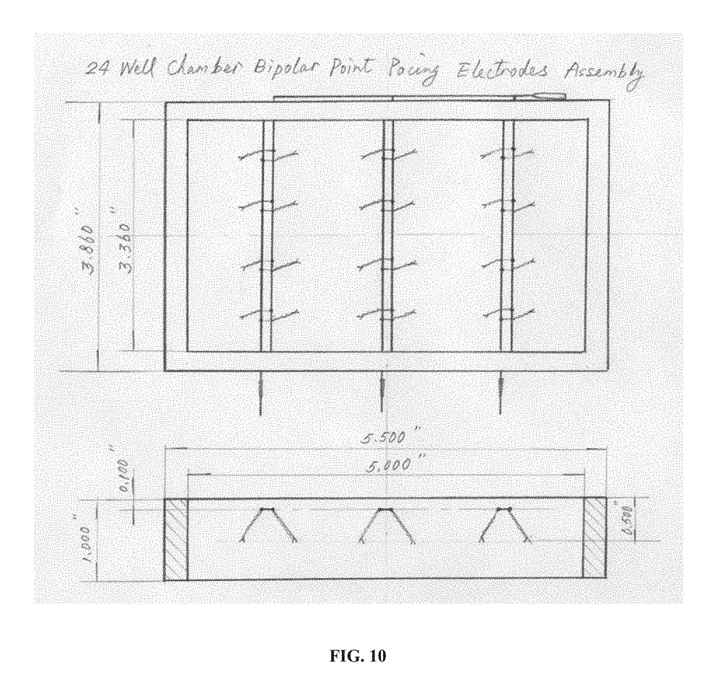

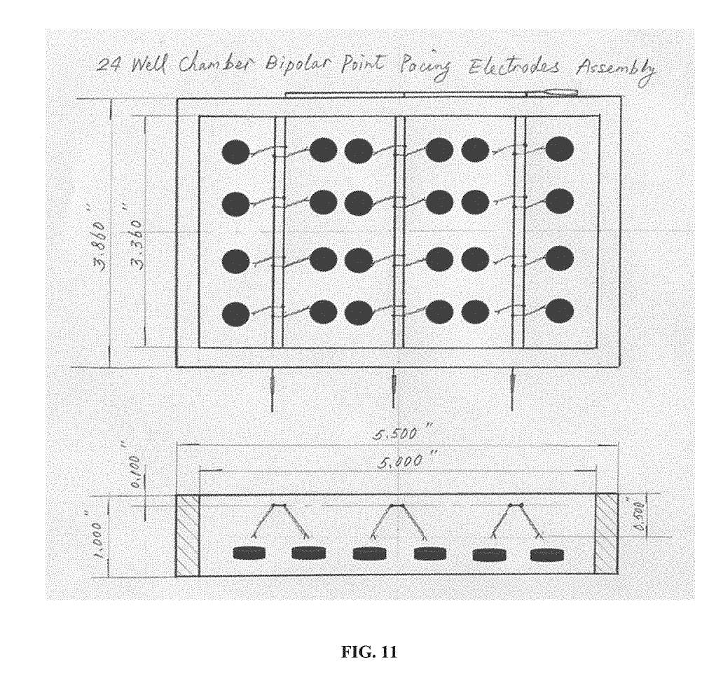

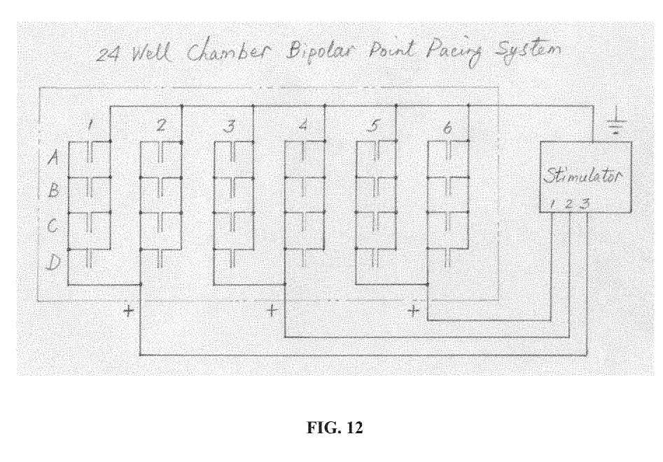

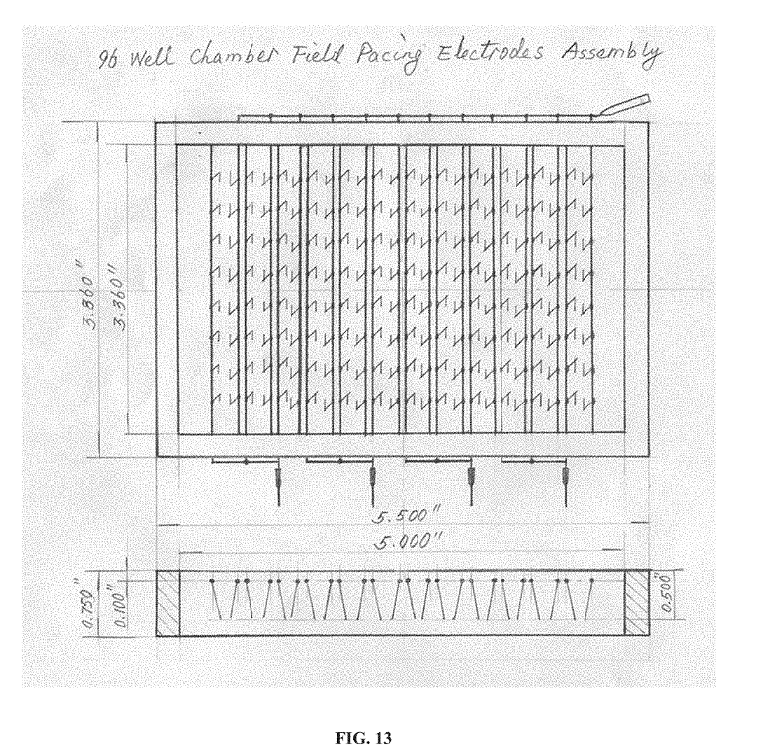

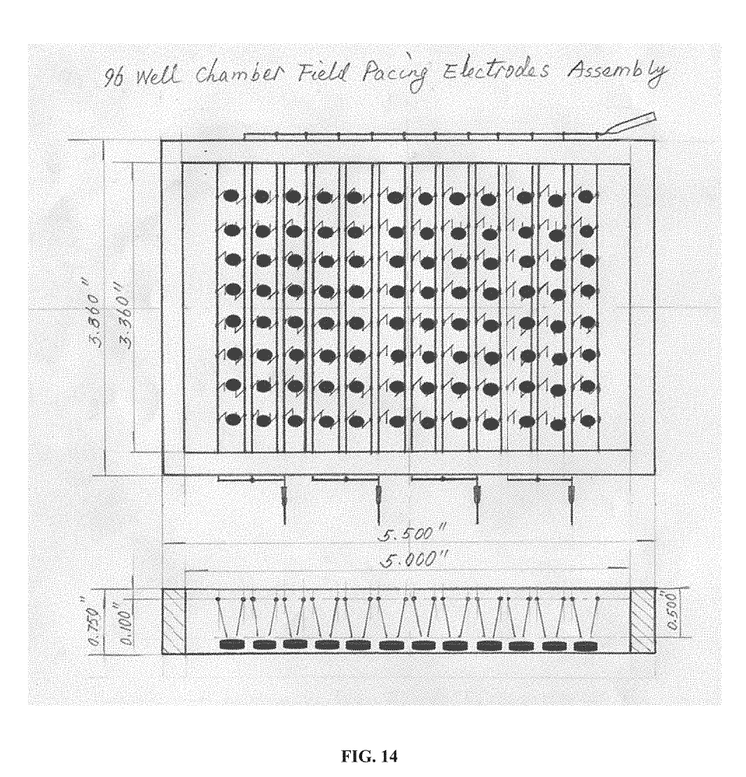

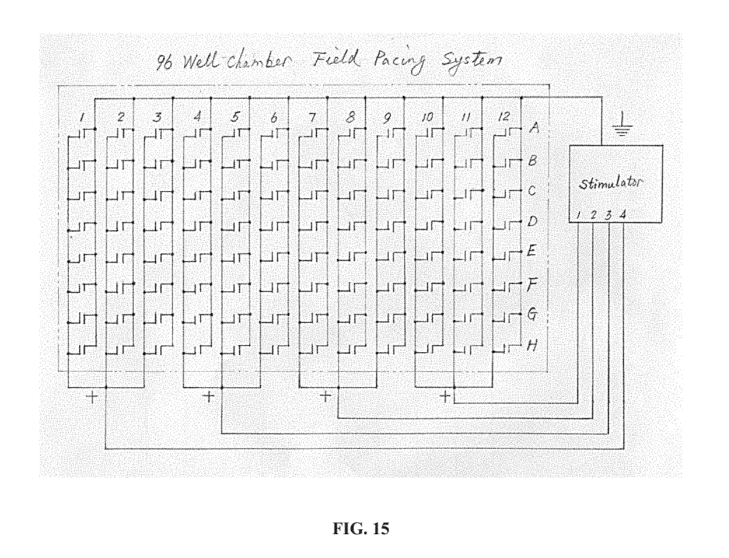

In some embodiments, the electrode array comprises 12 pairs of electrodes (see, e.g., FIGS. 1-6). In some embodiments, the electrode array comprises 24 pairs of electrodes (see, e.g., FIGS. 7-12). In some embodiments, the electrode array comprises 96 pairs of electrodes (see, e.g., FIGS. 13-15).

In some embodiments, the electrode array comprises a frame, said frame comprising and providing support for the electrodes. In some embodiments, the frame is built of plastic; in some embodiments, the electrodes are made from silver. In some embodiments, the electrode arrays (comprising a plastic frame and silver electrodes) provide electrical stimulation to cells in culture. During the development of embodiments of the technology provided herein, experiments indicated that electrical stimulation of human stem cell derived cardiomyocytes was successful in in each multi-well format tested (see, e.g., FIGS. 1-15). The technology is not limited in the stimulator that provides a current and/or voltage to the electrode array. Accordingly, any stimulator can be connected to the electrode frame.

In some embodiments, electrodes are arranged to provide field stimulation; in some embodiments, electrodes are arranged to provide point stimulation. Point stimulation is useful for measurement of impulse propagation velocity. Cells can be imaged using, e.g., an indicator substance such as, e.g., a fluorescent probe to report calcium flux, action potentials or any other physiological parameter.

### Multiwell Plates

The technology finds use with a variety of multiwell plates that are used to culture cardiomyocytes. As used herein, the term “multiwell plate” refers to one or more addressable wells located on a substantially flat surface. For instance, in some embodiments a multiwell plate comprises a two-dimensional array of addressable wells located on a substantially flat surface. Multiwell plates may comprise any number of discrete addressable wells, and comprise addressable wells of any width or depth. The Society for Biomolecular Screening has published recommended standard microplate specifications for a variety of plate formats (see, e.g., http://www.sbs-online.org); these recommended specifications are hereby incorporated herein by reference.

In some embodiments, wells are arranged in two-dimensional linear arrays on the multiwell plate. However, the wells can be provided in any type of array, such as in a geometric or in a non-geometric array. The multiwell plate can comprise any number of wells. Larger numbers of wells or increased well density can also be easily accommodated using the methods of the claimed invention. Commonly used numbers of wells include 6, 12, 96, 384, 1536, 3456, and 9600. In various embodiments, well volumes vary depending on well depth and cross sectional area. In some embodiments, the well volume is between approximately 0.1 microliters and 500 microliters. In various embodiments, wells have any cross sectional shape (e.g., in plan view) including square, round, hexagonal, other geometric or non-geometric shapes, and combinations (intra-well and inter-well) thereof. In some embodiments, the wells have a cross-sectional shape (e.g., in plan view) that is square or round with flat bottoms. In various embodiments, the walls of the wells are chamfered (e.g. having a draft angle). For instance, in some embodiments the angle is between approximately 1 and 10 degrees, between approximately 2 and 8 degrees, and in some embodiments between approximately 3 and 5 degrees. In various embodiments, the wells are placed in a configuration so that the well center-to well-center distance is between approximately 0.5 millimeters and approximately 100 millimeters. In various embodiments, the wells are placed in any configuration, such as a linear-linear array, or geometric patterns, such as hexagonal patterns.

For instance, the technology finds use in providing a stimulating current to pace cardiomyocytes cultured in, e.g., a 6-well, a 12-well (see, e.g., FIGS. 1-6), a 24-well (see, e.g., FIGS. 7-12), a 48-well, a 60-well, a 72-well, a 96-well (see, e.g., FIGS. 13-15), a 384-well, a 1536-well, and/or a 3456-well (“nanowell”) plate. In additional embodiments, the plate is custom made to have a defined number of wells (e.g., a number from 1 to approximately 5000, e.g., approximately 1, 10, 100, 1000, 2000, 3000, or up to 5000 wells and numbers of wells therebetween). In additional embodiments, the wells are arranged in any number of rows and/or columns or in any other geometrical configuration (e.g., a rectangular array, a hexagonal close-packed array (e.g., “honeycomb”), a series of concentric shapes (e.g., concentric circles), a single strip, a serpentine or zig-zag path, a spiral, etc.). In some embodiments, the multiwell plate comprises wells in a single row and/or a single column, e.g., an 8-well strip.

The technology is not limited in the diameter of the wells that are present in the multiwell plate. For example, some embodiments comprise wells having a diameter of, e.g., approximately 1 mm to 100 mm, e.g., 5 mm, 6 mm, 10 mm, 13 mm, 14 mm, 20 mm, 50 mm, 75 mm, 100 mm. In some embodiments, all wells of the multiwell plate have the same diameter and in some embodiments one or more wells of the multiwell plate have a different diameter than one or more other wells of the multiwell plate.

In some embodiments, one or more wells of the multiwell plate comprises an upper compartment separated from a lower compartment by a microporous membrane, e.g., one or more wells comprises a permeable support, e.g., a cell culture insert, e.g., as is commercially available from CORNING as a TRANSWELL permeable support. The technology is not limited in the material used for the permeable support, e.g., in some embodiments the permeable support is made from one or more of polycarbonate, polyester, polyethylene terephthalate, polytetrafluoroethylene, etc.

The technology is not limited in the geometry of the multiwell plate. In some embodiments, one or more wells of the multiwell plate has a flat bottom, a v-shaped (e.g., conical) bottom, a round bottom.

In various embodiments, the multiwell plate is made from an electrically non-conductive material and, in some embodiments, comprises an optically opaque material that interferes with the transmission of radiation, such as light, through the wall of a well or bottom of a well. Such optically opaque materials reduce the background associated with optical detection methods. Optically opaque materials comprise or include any material, such as dyes, pigments, or carbon black. The optically opaque material prevents radiation from passing from one well to another, to prevent cross-talk between wells, so that the sensitivity and accuracy of assays are increased. In some embodiments, the optically opaque material is reflective, e.g., in some embodiments the material comprises a thin metal layer, mirror coating, or a mirror polish. In some embodiments, optically opaque materials are coated onto any surface of the multiwell plate; in some embodiments the optically opaque material is an integral part of the plate or bottom as they are manufactured. In some embodiments, the optically opaque material prevents the transmittance of between approximately 100% to approximately 50% of incident light; in some embodiments between approximately 80% and greater than 95%; in some embodiments, more than 99%.

In some embodiments, one or more wells of the multiwell plate has a clear bottom; in some embodiments, one or more wells of the multiwell plate has a translucent bottom; in some embodiments, one or more wells of the multiwell plate has an opaque bottom.

The technology is not limited in the material used for construction of the multiwell plate provided that the material is compatible with using voltage to pace cardiomyocytes in the wells of the plate and/or to electrically isolate (e.g., insulate) each well from one or more other wells. The materials for manufacturing the multiwell plate will typically be polymeric, since these materials lend themselves to mass manufacturing techniques. Thus, in some embodiments, the multiwell plate is made from polystyrene, glass, quartz, polycarbonate, polyester, polyethylene terephthalate, polytetrafluoroethylene, etc.

In some embodiments, one or more wells of the multiwell plate is coated, e.g., with collagen, poly-d-lysine, etc.

Operation of the Electrode Array with a Multiwell Plate

In some embodiments, the electrode array provides a pair of electrodes to a well of a multiwell plate. In some embodiments, the well comprises a culture medium, e.g., a culture medium comprising a cardiomyocyte. Accordingly, in some embodiments the electrode array provides a current and/or voltage (e.g., a low-impedance current and/or voltage) to a cardiomyocyte in a well of a multiwell plate. In some embodiments, the electrode array provides a pair of electrodes (e.g., as a unipolar or bipolar arrangement) to each well of a multiwell plate, e.g., in some embodiments, the electrode array comprises from 1 pair of electrodes to approximately 5000 pairs of electrodes, e.g., approximately 1, 10, 100, 1000, or 5000 pairs of electrodes and numbers of pairs of electrodes therebetween. In some embodiments, the electrode array comprises 6 pairs of electrodes, 12 pairs of electrodes, 24 pairs of electrodes, 48 pairs of electrodes, 60 pairs of electrodes, 72 pairs of electrodes, 96 pairs of electrodes, 384 pairs of electrodes, 1536 pairs of electrodes, and/or 3456 pairs of electrodes, e.g., to provide a current and/or a voltage to one or more wells of a 6-well, a 12-well, a 24-well, a 48-well, a 60-well, a 72-well, a 96-well, a 384-well, a 1536-well, and/or a 3456-well plate. In some embodiments, the electrode array comprises 6 bipolar electrodes, 12 bipolar electrodes, 24 bipolar electrodes, 48 bipolar electrodes, 60 bipolar electrodes, 72 bipolar electrodes, 96 bipolar electrodes, 384 bipolar electrodes, 1536 bipolar electrodes, and/or 3456 bipolar electrodes, e.g., to provide a current and/or a voltage to one or more wells of a 6-well, a 12-well, a 24-well, a 48-well, a 60-well, a 72-well, a 96-well, a 384-well, a 1536-well, and/or a 3456-well plate.

The electrode array provides the current and/or voltage to the wells of the multiwell plate while allowing access to the multiwell plate, e.g., to provide a test treatment (e.g., a test substance) to one or more wells, to illuminate one or more wells with a light source (e.g., a fluorescence excitation wavelength, visible light, etc.), to image one or more cells and/or one or more wells (e.g., using a charge-coupled device camera), to record an action potential of one or more cells (e.g., using one or more recording microelectrodes), to record an ion channel current (e.g., potassium and/or sodium channel), and/or to record a fluorescence emission from one or more wells, etc. In some embodiments, voltage and/or current is provided to one or more wells manually. In some embodiments, voltage and/or current is provided to one or more wells using an automated system (e.g., software and hardware components). In some embodiments, voltage and/or current is provided to one or more cardiomyocytes using an automated system (e.g., software and hardware components), e.g., to control the rate of pacing (e.g., cardiomyocyte beat rate) of the one or more cardiomyocytes.

In some embodiments, providing a test treatment to one or more wells, illuminating one or more wells with a light source, imaging one or more wells, recording an action potential of one or more cells (e.g., using one or more recording microelectrodes), recording an ion channel current (e.g., potassium and/or sodium channel), and/or recording fluorescence emission from one or more cells is performed manually. In some embodiments, providing a test treatment to one or more wells, illuminating one or more wells with a light source, imaging one or more wells, recording an action potential of one or more cells (e.g., using one or more recording microelectrodes), recording an ion channel current (e.g., potassium and/or sodium channel), and/or recording fluorescence emission from one or more cells is performed using an automated system, e.g., using a computer, software, wires, robotics, analog-to-digital converters, digital-to-analog converters, etc. to control providing a test treatment to one or more wells, illuminating one or more wells with a light source, imaging one or more wells, recording an ion channel current (e.g., potassium and/or sodium channel), recording an action potential of one or more cells (e.g., using one or more recording microelectrodes), and/or recording fluorescence emission from one or more cells using a computer and software.

### Cells

The technology is not limited in the cells that are cultured and exposed to a current and/or a voltage by the electrode array. Accordingly, in various embodiments many types of cells (e.g., cultured cells, stem cells, synthetic cells) are employed with the technology described herein. In some embodiments, the cell is a pluripotent cell with potential for cardiomyocyte differentiation. Such cells include embryonic stem cells and induced pluripotent stem cells, regardless of source. For example, induced pluripotent stem cells may be derived from stem cells or adult somatic cells that have undergone a dedifferentiation process.

Exemplary cells that are studied using embodiments of the technology include cardiomyocytes, neurons, stem cell-derived cardiomyocytes, stem cell-derived neurons, cells comprising ion channels, cells comprising a proton pump, etc.

Induced pluripotent stem cells (iPSCs) may be generated using any known approach. In some embodiments, iPSCs are obtained from adult human cells (e.g., fibroblasts). In some embodiments, modification of transcription factors (e.g., Oct3/4, Sox family members (Sox2, Sox1, Sox3, Sox15, Sox18), Klf Family members (Klf4, Klf2, Klf1, Klf5), Myc family members (c-myc, n-myc, 1-myc), Nanog, LIN28, Glis1, etc.), or mimicking their activities is employed to generate iPSCs (e.g., using a transgenic vector (adenovirus, lentivirus, plasmids, transposons, etc.), inhibitors, delivery of proteins, microRNAs, etc.).

In some embodiments, the cells are stem cell-derived cardiomyocytes produced using compositions and methods described in U.S. Pat. App. Pub. No. 2015/0329825, incorporated herein by reference in its entirety.

In some embodiments, the cells are non-terminally differentiated cells (regardless of pluripotency) or other non-maturated cells.

In some embodiments, cells are screened for propensity to develop teratomas or other tumors (e.g., by identifying genetic lesions associated with a neoplastic potential). Such cells, if identified, are discarded.

In some embodiments, the cells are stem cell-derived cardiomyocytes. For example, in some embodiments stem cells are cultured and/or differentiated on a flexible (e.g., soft, pliable) surface (e.g., polydimethylsiloxane) coated with ECM proteins. In some embodiments, the stem cell-derived cardiomyocytes are as described in U.S. Pat. App. Pub. No. 2015/0329825, herein incorporated by reference in its entirety.

In some embodiments, cells are modified to include a marker and used as diagnostic compositions to assess properties of the cells in response to changes in their environment.

### Culturing/Differentiation

Culture conditions are selected based on the cells employed. In some embodiments, the conditions used are those of Lee et al. (Circulation Research 110: 1556-63 (2012), herein incorporated by reference in its entirety). In some embodiments, the process comprises thawing (if cryopreserved) and plating iPSCs on a coated support at a desired density (e.g., 125,000 cells per monolayer) in differentiation media (e.g., embryoid body differentiation media, commonly referred to as embryoid body-20, comprising 80% Dulbecco Modified Eagle Medium (DMEM/F12), 0.1 mmol/L⁻¹ nonessential amino acids, 1 mmol/L⁻¹ L-glutamine, 0.1 mmol/L⁻¹ β-mercaptoethanol, and 20% fetal bovine serum; Gibco) supplemented with 10 μmol/L⁻¹ blebbistatin. In some embodiments, after 24 hours in embryoid body-20, the medium is switched to iCell maintenance medium (Cellular Dynamics), supplemented with 10 μmol/L⁻¹ blebbistatin, and cells are cultured for an additional time period (e.g., 96 hours) at 37° C., in 5% CO₂, with the medium changed once daily. See, e.g., culture conditions described in U.S. Pat. App. Pub. No. 2015/0329825, herein incorporated by reference in its entirety.

Some methods for cardiomyocyte isolation, culture, and genetic manipulation are provided, e.g., in J Mol Cell Cardiol. 2011 September; 51(3): 288-298, incorporated herein by reference in its entirety.

### Methods

Some embodiments of the technology relate to methods of applying a voltage and/or a current to one or more cells (e.g., cardiomyocytes) in one or more wells of a multiwell plate, e.g., in parallel.

Some embodiments relate to methods of assessing the effects of one or more agents (e.g., test compounds, e.g., drugs, etc.) on one or more cells (e.g., cardiomyocytes) in one or more wells of a multiwell plate, e.g., to provide a high-throughput (e.g., multiplex, highly parallel) assay of one or more test agents and/or conditions on a cell (e.g., a cardiomyocyte). Methods include one or more steps such as, e.g., providing an electrode array device as described herein, providing a multiwell plate, providing a culture medium (e.g., a conductive culture medium) to a well of a multiwell plate, providing a cell to a well of a multiwell plate, contacting the culture medium with a pair of electrodes from the electrode array device, providing a current and/or a voltage to a well and/or a cell in a well of a multiwell plate, adding a test agent to a well and/or to a well comprising a cell, recording data, e.g., by observing a well of a multiwell plate (e.g., imaging a well of a multiwell plate (e.g., in some embodiments, optical mapping as described in U.S. Pat. App. Pub. No. 2015/0329825, incorporated herein by reference in its entirety), e.g., using a charge coupled device camera; recording an emission spectrum emission at a particular wavelength or range of wavelengths; recording a fluorescence emission spectrum or fluorescence emission at a particular wavelength or range of wavelengths; recording an emission spectrum emission at a particular wavelength or range of wavelengths as a function of time; recording a fluorescence emission spectrum or fluorescence emission at a particular wavelength or range of wavelengths as a function of time; recording a voltage; recording a voltage as a function of time; measuring the concentration of an ion inside or outside the cell; measuring the concentration of an ion inside or outside the cell as a function of time; etc.).

Some embodiments comprise varying the electrical stimulation (e.g., a voltage and/or a current) provided to the electrode array, e.g., to provide a series of electrical stimulation pulses and/or signals, e.g., to provide a series of current and/or voltage pulses to one or more wells of a multiwell plate and, in some embodiments, to provide a series of current and/or voltage pulses to one or more samples present in the one or more wells of the multiwell plate. In some embodiments, the electrical stimulation (e.g., the current and/or voltage) is varied according to a function such as, e.g., a square wave, a sinusoidal wave, a triangle wave, or any other wave shape.

In some embodiments, the methods comprise illuminating one or more wells and/or cells with a particular wavelength or range of wavelengths of electromagnetic radiation, e.g., providing an excitation wavelength. Some embodiments comprise culturing cells (e.g., cardiomyocytes) in a well of a multiwell plate. In some embodiments, culturing cells is as described in U.S. Pat. App. Pub. No. 2015/0329825, incorporated herein by reference in its entirety. Some embodiments comprise recording an action potential of one or more cells (e.g., cardiomyocytes), e.g., using a microelectrode (e.g., as described in U.S. Pat. App. Pub. No. 2015/0329825, incorporated herein by reference in its entirety). Some embodiments comprise recording a voltage and/or a current of one or more cells (e.g., cardiomyocytes), e.g., using a patch clamp method (e.g., as described in U.S. Pat. App. Pub. No. 2015/0329825, incorporated herein by reference in its entirety).

Steps of the methods are performed manually in some embodiments. Steps of the methods are automated in some embodiments (e.g., performed using machines, robots, etc., e.g., under control of a computer and/or software and/or comprising using electromechanical interfaces, analog-to-digital converters, digital-to-analog converters, etc. to control devices and/or apparatuses using embodiments of methods implemented in software).

In some embodiments, cardiomyocytes are provide with an in vitro environment and stimulated with electrical signals that are similar to the in vivo environment and electrical signals experienced by cardiomyocytes in vivo. For example, in some embodiments, a culture medium is used that has a high conductivity, e.g., a conductivity of approximately 5 to 20 mS/cm (e.g., 5, 6, 7, 8, 9, 10, 11, 12, 13, 14, 15, 16, 17, 18, 19 or 20 mS/cm), which is similar to the conductivity that of extracellular fluid that surrounds living cells in vivo (e.g., 3-12 mS/cm; see, e.g., Durand “Electric stimulation of excitable tissue”, in: Durand and Bronzino, eds. The Biomedical Engineering Handbook. CRC Press, Boca Raton, Fla. (1995), pp. 229-51). Furthermore, in some embodiments an electrical field is used (e.g., applied to cells, e.g., cardiomyocytes) that is in the physiologically range that cardiomyocytes experience in vivo, e.g., approximately 0.1 to 10 V/cm (e.g., 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 and values and ranges therebetween (see, e.g., Song et al. (2007) “Application of direct current electric fields to cells and tissues in vitro and modulation of wound electric field in vivo” Nat Protoc 2: 1479-89). In some embodiments, the cardiomyocytes are stimulated to beat similarly to cardiomyocytes in vivo. In particular, an animal heart beats from approximately 3 beats per minute (e.g., whales during dives) up to approximately 360 beats per minute (rat) and approximately 1,200 beats per minute for a hummingbird during flight, which is a frequency range corresponding to 0.05-20 Hz. A normal resting heart rate for human adults ranges from approximately 60 to 100 beats a minute. A lower heart rate typically implies more efficient heart function and better cardiovascular fitness, e.g., a well-trained athlete may have a normal resting heart rate near 40 beats a minute. In some embodiments, cardiomyocytes are pulsed with a pulse lasting approximately 1 to 2 ms, which is sufficiently long to excite heart tissue and cells. In some embodiments, cardiomyocytes are stimulated with an electrical signal that comprises, e.g., a monophasic pulses, charge-balanced biphasic pulses, charge-balanced biphasic pulses with interphase delay, charge-balanced biphasic pulses with slow reversal, and/or direct current.

### Systems

Embodiments of the technology also provide systems for automated pacing of cardiomyocytes and performing assays using the cardiomyocytes. In some embodiments, the technology comprises systems for automated pacing of cardiomyocytes and performing assays using the cardiomyocytes and a spectroscopic or fluorimetric or electrical measurement. Exemplary embodiments of systems comprise one or more of, e.g., an electrode array, a component for generating a current and/or voltage (e.g., to supply the electrodes of the electrode array with a voltage and/or a current, e.g., a generator, a battery), a source of electromagnetic radiation (e.g., ultraviolet, visible, infrared), a detector of electromagnetic radiation (e.g., ultraviolet, visible, infrared) (e.g., a camera, e.g., a CCD camera), an analog-to-digital (A/D) converter, a digital-to-analog (D/A) converter, a computer and software to coordinate the generation of electrical current and/or voltage, collection of data, and movement of multiwell plates. In some embodiments, the system can further comprise a component for addition of a fluid (e.g., a composition comprising one or more test compounds or test treatments) to one or more wells of the multiwell plate. In some embodiments, the systems find use in modulating, characterizing, and assaying cardiomyocytes and the effects of a test compound on cardiomyocytes, e.g., for rapidly screening the effects of test compounds on cardiomyocytes. Some embodiments further comprise a function or wave generator to provide a series of pulses and/or signals, e.g., to provide a series of current and/or voltage pulses to one or more wells of a multiwell plate. In some embodiments, the function or wave generator provides a wave that is, e.g., a square wave, a sinusoidal wave, a triangle wave, or any other wave shape. In some embodiments, the systems further comprise a power amplifier, a transformer, and/or a display.

In some embodiments, the technology comprises an optogenetic component and/or an optogenetic system. For example, embodiments provide technology for characterizing a cell by incorporating into a cell (e.g., a cardiomyocyte) an optical actuator of electrical activity and an optical reporter of electrical activity. In some embodiments, a cell (e.g., a cardiomyocyte) receives the actuator and/or the reporter. In certain embodiments, a cell will receive both via transfection with a single vector that includes genes coding for each of the reporter and actuator. As used herein, the term “optical reporter” refers to a structure or system employed to yield an optical signal indicative of cellular electrical or neural activity such as a voltage drop across a membrane, a synaptic transmission, an action potential, a release or uptake or non-uptake of a neurotransmitter, etc. As used herein, the term “membrane potential” refers to a calculated difference in voltage between the interior and exterior of a cell. In certain embodiments, an optical reporter of electrical activity in a cell is provided by a microbial rhodopsin or a modified microbial rhodopsin. A typical microbial rhodopsin is a light-driven proton pump structured as an integral membrane protein belonging to the family of archaeal rhodopsins. In some embodiments, the microbial rhodopsins or modified microbial rhodopsins are used as an optically detectable sensor to sense voltage across membranous structures, such as in cells (e.g., a cardiomyocyte) when they are present in the lipid bilayer membrane. That is, the microbial rhodopsin proteins and the modified microbial rhodopsin proteins are used in some embodiments as optical reporters to measure changes in membrane potential of a cell, including prokaryotic and eukaryotic cells (e.g., a cardiomyocyte). In some embodiments, an optical reporter is used in methods for drug screening, in research settings, and in in vivo imaging systems.

Exemplary microbial rhodopsins include: green-absorbing proteorhodopsin (GPR, Gen Bank #AF349983), a light-driven proton pump found in marine bacteria; blue absorbing proteorhodopsin (BPR, GenBank #AF349981), a light-driven proton pump found in marine bacteria; Natronomonas pharaonis sensory rhodopsin II (NpSR11, GenBank #Z35086.1), a light-activated signaling protein found in the halophilic bacterium N. pharaonis; bacteriorhodopsin (BR, GenBank #NC010364.1), a light-driven proton pump found in Halobacterium salinarurm; Archaerhodopsin 3 (Arch3, GenBank #P96787), a light-driven proton pump found in Halobacterium sodomense; variants of the foregoing, and others discussed herein. Additional rhodopsions that can be mutated as indicated in the methods of the invention include fungal opsin related protein (Mac, GenBank #AAGO1180); Cruxrhodopsin (Crux, GenBank #BAA06678); Algal bacteriorhodopsin (Ace, GenBank #AAY82897); Archaerhodopsin 1 (Arch 1, GenBank #P69051); Archaerhodopsin 2 (Arch 2, GenBank #P29563); and Archaerhodopsin 4 (Arch 4, GenBank #AAG42454). Some of the foregoing are pointed to by Genbank number. However, a rhodopsin may vary from a sequence in GenBank. Based on the description of the motif described herein, a skilled artisan will easily be able to make homologous mutations in microbial rhodopsin genes to achieve the described or desired functions, e.g. reduction in the pumping activity of the microbial rhodopsin in question.

### Uses

The electrode array technology finds use in many applications. For example, in some embodiments the technology finds use in cardiotoxicity testing, e.g., using chronic electrical pacing of cardiomyocytes in culture. In some embodiments, the technology finds use in cardiovascular research, e.g., to study genetic and environmental factors that modulate cardiomyocyte and heart physiology. The technology is not limited with respect to the types of cells that can be studied, e.g., the technology finds use in studying any natural and/or synthetic cell that reacts and/or can be modulated by application of a current or a voltage to the cell. Exemplary cells that are studied using embodiments of the technology include cardiomyocytes, neurons, stem cell-derived cardiomyocytes, stem cell-derived neurons, cells comprising ion channels, cells comprising a proton pump, etc. In some embodiments, the technology finds use in studying calcium flux, e.g., through a calcium channel. In some embodiments, the technology finds use in studying sodium flux, e.g., through a sodium channel. In some embodiments, the technology finds use in studying proton flux, e.g., through a proton channel or pump. In some embodiments, the technology finds use in studying the generation of reactive oxygen species generation. In some embodiments, the technology finds use in monitoring cell voltage. In some embodiments the technology uses a fluorescent dye as a signal indicative of calcium concentration, calcium flux, reactive oxygen species, or other analytes of interest.

Also provided herein are methods, devices, and systems for using such cells for research, drug screening, diagnostic, and therapeutic applications. For example, in some embodiments, provided herein are high-throughput screening methods, devices and systems that permit multiple of such cell compositions to be tested in parallel (e.g., in a device comprising a plurality of chambers (e.g., wells) containing such cells). In some embodiments, a membrane comprising such cells is provided, for example, for transplantation to a subject for research, diagnostic, or therapeutic purposes. In some embodiments, screening methods comprise: a) providing a cell composition described above; b) exposing a test compound (e.g., a candidate therapeutic compound) to the composition; and c) determining an effect of the test compound on said composition. In some embodiments, the effect is one or more cardiac electrophysiological functions including, but not limited to, action potential duration, beating frequency, conduction velocity or intracellular calcium flux amplitudes.

In some embodiments, the technology finds use for disease modeling and drug development. In some embodiments, the technology is used in combination with cardiomyocytes as described in U.S. Pat. App. Pub. No. 2015/0329825, herein incorporated by reference in its entirety. The electrode array technology and high-quality cells described therein provide a technology appropriate for research uses, particularly using high-throughput analysis. For example, in some embodiments agents (e.g., antiarrhythmic agents) are contacted with the cells to determine the effect of the agent on the cells, e.g., the cell's response to a voltage and/or current provide by one or more electrodes of the electrode array in the presence of the agent provided to contact the cell.

In some embodiments, the electrode array is use for drug testing applications. For example, in some embodiments, drugs or biological agents are added to cardiomyocytes (e.g., pacing cardiomyocytes) and tested with respect to the effects on the electrophysiological characteristics of the cardiomyocytes (e.g., to assess the effects of the test compounds on the quality and/or quantity of pacing of the cardiomyocytes). Indications for drug testing include any compound or biological agent in the pharmaceutical discovery and development stages, or drugs approved by drug regulatory agencies, such as the US Federal Drug Agency. The technology finds use with all classes of drugs, including prescription drugs, over-the-counter drugs, biologics, biosimilars, and nutraceutical agents, for any medical indications, including but not limited to, drugs for treating cancer, neurological disorders, fertility, vaccines, blood pressure, blood clotting, and immunological disorders; further examples of drugs include but are not limited to anti-infectives, anti-fungals, anti-allergens, antibiotics, steroids, anti-inflammatories, and drugs for cardiovascular-related disorders.

In some embodiments, drug testing applications determine the effects of new chemical entities on cardiac electrophysiological function including, but not limited to, action potential duration, beating frequency, conduction velocity, and intracellular calcium flux amplitudes. Such assays serve to inform drug development businesses on the risk of a compound to cause fatal cardiac arrhythmia or other heart-related side effects. These tests may be acute or performed following long term exposure to a drug.

Embodiments of the present technology provide kits comprising an electrode array device described herein. In some embodiments, kits further comprise one or more multiwell plates and/or culture medium. In some embodiments, kits further comprise cells described in U.S. Pat. App. Pub. No. 2015/0329825, herein incorporated by reference in its entirety. In some embodiments, kits comprise an electrode array device, cells (e.g., cardiomyocytes or iPSC or stem cells suitable for differentiating into cardiomyocytes) in or on a flexible surface (e.g., multi-well plate or other surface), and reagents for differentiation or use of cells (e.g., buffers, test compounds, controls, etc.).

1-35. (canceled)

36. An electrode array that provides electrical current or voltage to a plurality of samples in a multiwell plate, the electrode array comprising a plurality of electrodes or electrode pairs, wherein each electrode or electrode pair provides a current or a voltage to a single well of a multiwell plate.

37. The electrode array of claim 36 wherein each electrode or electrode pair of the electrode array provides field stimulation to a sample in a well of the multiwell plate and/or provides point stimulation to a sample in a well of the multiwell plate.

38. The electrode array of claim 36 wherein each electrode or electrode pair of the electrode array is present in a low impedance circuit.

39. The electrode array of claim 36 wherein said well of the multiwell plate comprises a sample comprising a cell, tissue, organ, or organoid.

40. The electrode array of claim 36 wherein said well of the multiwell plate comprises a sample comprising a material that responds to electrical stimulation.

41. The electrode array of claim 36 wherein said well of the multiwell plate comprises a sample comprising a cardiomyocyte.

42. The electrode array of claim 36 wherein the electrode array comprises 12, 24, or 96 electrodes or electrode pairs.

43. A system for providing electrical stimulation to a plurality of samples, the system comprising:
a) an electrode array that provides electrical current or voltage to a plurality of samples in a multiwell plate, the electrode array comprising a plurality of electrodes or electrode pairs, wherein each electrode or electrode pair provides a current or a voltage to a single well of a multiwell plate; and
b) a multiwell plate.

44. The system of claim 43 wherein the electrode array is adapted to fit over the multiwell plate such that each electrode or electrode pair provides a current or a voltage to a well of the multiwell plate.

45. The system of claim 43 further comprising a source of stimulating current or voltage.

46. The system of claim 43 further comprising a dye for indicating changes of a sample in a well of the multiwell plate.

47. The system of claim 43 further comprising a source of electromagnetic radiation providing an excitation wavelength and a detector of an emission wavelength of electromagnetic radiation.

48. The system of claim 43 further comprising a software or hardware component for controlling a stimulating current or voltage.

49. A method of providing electrical stimulation to one or more samples in parallel, the method comprising:
a) providing one or more samples in one or more wells of a multiwell plate;
b) contacting one or more wells of the multiwell plate with an electrode array that provides electrical current or voltage to the one or more samples in the multiwell plate, the electrode array comprising a plurality of electrodes or electrode pairs, wherein each electrode or electrode pair provides a current or a voltage to a single well of a multiwell plate; and
c) providing electrical stimulation to the one or more samples via the electrode array.

50. The method of claim 49 further comprising observing the one or more samples stimulated by electrical stimulation.

51. The method of claim 49 further comprising contacting one or more samples with an indicator substance.

52. The method of claim 49 further comprising illuminating one or more samples with an excitation wavelength of electromagnetic radiation and monitoring one or more samples for an emission wavelength of electromagnetic radiation.

53. The method of claim 14 further comprising varying the electrical stimulation provided to the electrode array.

54. The method of claim 49 wherein one or more samples comprises a cardiomyocyte and the method comprising pacing the cardiomyocyte.

55. The method of claim 49 further comprising varying the electrical stimulation provided to the electrode array at a frequency of 60 to 100 pulses per minute.
